(19) United States
(12) Patent Application Publication
(10) Pub. No.: US 2024/0050656 A1
(43) Pub. Date: Feb. 15, 2024

(54) OCCLUSION DETECTION SYSTEM AND METHOD FOR A FLOW CONTROL APPARATUS

(71) Applicant: KPR U.S., LLC, Mansfield, MA (US)

(72) Inventors: Meghan FOX, St. Louis, MO (US); Joseph HUDSON, O'Fallon, MO (US); Lara TAPY, Chesterfield, MO (US); Andrew DUNN, Mansfield, MA (US)

(21) Appl. No.: 18/447,698

(22) Filed: Aug. 10, 2023

Related U.S. Application Data
(60) Provisional application No. 63/396,882, filed on Aug. 10, 2022.

(51) Int. Cl. A61M 5/168; A61J 15/00

(52) U.S. Cl. CPC A61M 5/16886; A61J 15/0076; A61M 2205/3334

(57) ABSTRACT

A flow control system and method of detecting a condition of a pump set mounted on the apparatus. The system configured to perform the method of advancing a first amount of the feeding formula through the feeding pump via a feeding formula conduit. Further, the system may acquire a first pressure sensor reading of the feeding formula conduit. Further, the system may advance a second amount of the feeding formula through the feeding pump via the feeding formula conduit, wherein the second amount is second volume of the liquid. Further, the system may acquire a second pressure sensor reading of the feeding formula conduit, and acquire a third pressure sensor reading of the feeding formula conduit. Further, the system may detect an occlusion in the feeding formula conduit based on at least one of the first pressure sensor reading, the second pressure sensor reading and the third pressure sensor reading.

### CROSS-REFERENCE TO RELATED APPLICATION

This application claims priority to U.S. Provisional Patent Application No. 63/396,882, entitled “OCCLUSION DETECTION SYSTEM AND METHOD FOR A FLOW CONTROL APPARATUS,” filed on Aug. 10, 2022, which is hereby incorporated by reference in its entirety.

### TECHNICAL FIELD

The present disclosure generally relates to a flow control system and method capable of detecting a condition of a pump set mounted on the apparatus. More specifically, occlusion detection for thick formula feeds.

### BACKGROUND

Administering fluids containing medicine or nutrition to a patient is generally known in the art. Typically, fluid is delivered to the patient by a pump set received by a flow control apparatus, such as a pump, connected to a source of fluid which delivers fluid to a patient. A flow control apparatus may be capable of monitoring and detecting fluid flow conditions that can occur within the loaded administration feeding set during operation of the flow control apparatus. Generally, flow monitoring systems that are capable of monitoring and detecting flow conditions may rely on sensors arranged relative to the administration feeding set.

### SUMMARY

The following presents a simplified summary of one or more implementations of the present disclosure in order to provide a basic understanding of such implementations. This summary is not an extensive overview of all contemplated implementations, and is intended to neither identify key or critical elements of all implementations nor delineate the scope of any or all implementations. Its sole purpose is to present some concepts of one or more implementations of the present disclosure in a simplified form as a prelude to the more detailed description that is presented later.

In one aspect, the disclosure provides a system, method and non-transitory computer readable medium storing computer executable instructions that may be executed by a processor for detecting the flow of a fluid via a control apparatus. The system, method and non-transitory computer readable medium may include a housing configured to receive a feeding set; a pumping device configured to produce a fluid flow in the feeding set; and a processor configured to determine an occlusion in the fluid flow based on a sensor detection; wherein the fluid flow is of thick formula.

In some aspects, the techniques described herein relate to a method of occlusion detection of a feeding pump, the method including: determining when a feeding set including a liquid is engaged in the feeding pump, wherein the liquid is a feeding formula; advancing a first amount of the feeding formula through the feeding pump via a feeding formula conduit, wherein the first amount is a first volume of the liquid; acquiring a first pressure sensor reading of the feeding formula conduit; advancing a second amount of the feeding formula through the feeding pump via the feeding formula conduit, wherein the second amount is second volume of the liquid; acquiring a second pressure sensor reading of the feeding formula conduit; acquiring a third pressure sensor reading of the feeding formula conduit; and detecting when an occlusion is present in the feeding formula conduit based on at least one of the first pressure sensor reading, the second pressure sensor reading and the third pressure sensor reading.

In some aspects, the techniques described herein relate to a feeding pump configured for occlusion detection including: one or more memories; and one or more processors coupled with the one or more memories and configured, individually or in combination, to: determine when a feeding set including a liquid is engaged in the feeding pump, wherein the liquid is a feeding formula; advance a first amount of the feeding formula through the feeding pump via a feeding formula conduit, wherein the first amount is a first volume of the liquid; acquire a first pressure sensor reading of the feeding formula conduit; advance a second amount of the feeding formula through the feeding pump via the feeding formula conduit, wherein the second amount is second volume of the liquid; acquire a second pressure sensor reading of the feeding formula conduit; acquire a third pressure sensor reading of the feeding formula conduit; and detect when an occlusion is present in the feeding formula conduit based on at least one of the first pressure sensor reading, the second pressure sensor reading and the third pressure sensor reading.

In some aspects, the techniques described herein relate to a computer-readable medium including stored instructions for occlusion detection, wherein the instructions are executable by one or more processors, individually or in combination, to: determine when a feeding set including a liquid is engaged in a feeding pump, wherein the liquid is a feeding formula; advance a first amount of the feeding formula through the feeding pump via a feeding formula conduit, wherein the first amount is a first volume of the liquid; acquire a first pressure sensor reading of the feeding formula conduit; advance a second amount of the feeding formula through the feeding pump via the feeding formula conduit, wherein the second amount is second volume of the liquid; acquire a second pressure sensor reading of the feeding formula conduit; acquire a third pressure sensor reading of the feeding formula conduit; and detect when an occlusion is present in the feeding formula conduit based on at least one of the first pressure sensor reading, the second pressure sensor reading and the third pressure sensor reading.

In some aspects, the techniques described herein relate to a feeding set configured for occlusion detection of a feeding pump including: a liquid, wherein the liquid is a feeding formula; and a feeding formula conduit, wherein the feeding pump is configured to advance a first amount of the feeding formula though the feeding formula conduit, and the first amount is a first volume of the liquid; wherein the feeding pump is further configured to: acquire a first pressure sensor reading of the feeding formula conduit; advance a second amount of the feeding formula through the feeding pump via the feeding formula conduit, wherein the second amount is second volume of the liquid; acquire a second pressure sensor reading of the feeding formula conduit; acquire a third pressure sensor reading of the feeding formula conduit; and detect when an occlusion is present in the feeding formula conduit based on at least one of the first pressure sensor reading, the second pressure sensor reading and the third pressure sensor reading.

Additional advantages and novel features relating to implementations of the present disclosure will be set forth in part in the description that follows, and in part will become more apparent to those skilled in the art upon examination of the following or upon learning by practice thereof.

### BRIEF DESCRIPTION OF THE DRAWINGS

The novel features believed to be characteristic of the disclosure are set forth in the appended claims. In the descriptions that follow, like parts are marked throughout the specification and drawings with the same numerals, respectively. The drawing figures are not necessarily drawn to scale and certain figures may be shown in exaggerated or generalized form in the interest of clarity and conciseness. The disclosure itself, however, as well as a preferred mode of use, further objects and advances thereof, will be best understood by reference to the following detailed description of illustrative aspects of the disclosure when read in conjunction with the accompanying drawings, wherein:

FIG. 1 is a perspective view of an example enteral feeding pump and a fragmentary portion of a feeding set (feed/flush) received on the pump in accordance with aspects of the present disclosure;

FIG. 2 is a perspective view of FIG. 1 with a cassette housing of the feeding set removed;

FIG. 3 is the perspective view of FIG. 2 with the feeding set removed;

FIG. 4 is a block diagram illustrating elements of an example enteral feeding pump including a flow monitoring system in accordance with aspects of the present disclosure;

FIG. 5 is a flowchart of an example method of an occlusion detection routine in accordance with an implementation of the present disclosure;

FIG. 6 is an example block diagram of various hardware components and other features of a computer system that may operate the access control system in accordance with aspects of the present disclosure;

FIG. 7 is a block diagram of various example system components, for use in accordance with aspects of the present disclosure; and

FIGS. 8A-8N illustrate example results associated with the occlusion detection routine in accordance with an implementation of the present disclosure.

Corresponding reference characters indicate corresponding parts throughout the drawings.

### DETAILED DESCRIPTION

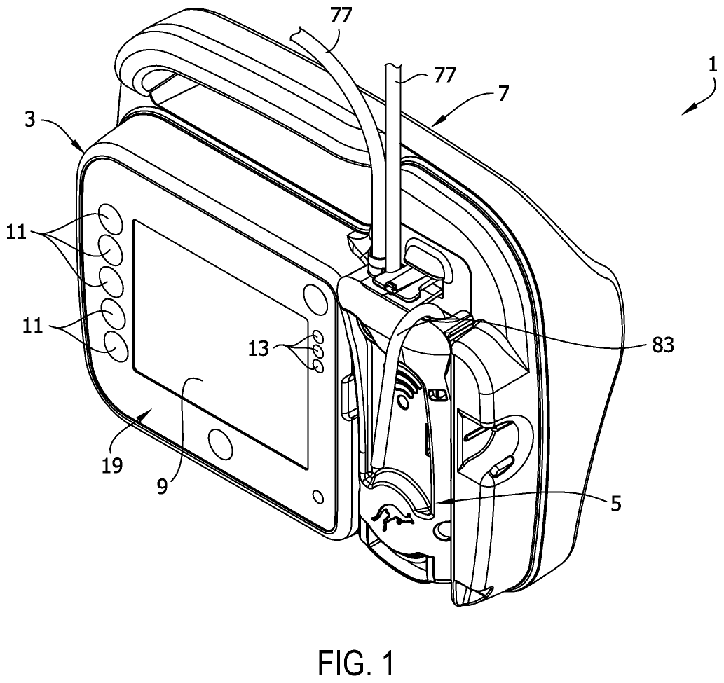

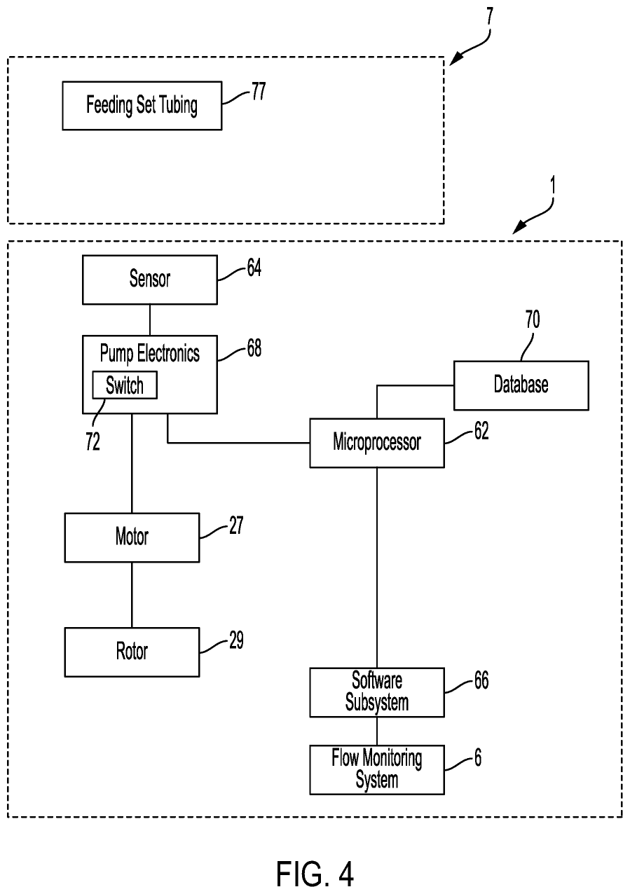

Referring now to the example aspects schematically illustrated in FIGS. 1-3, an enteral feeding pump (broadly, “a flow control apparatus”) and hereinafter interchangeably referred to throughout this disclosure as a “pump” is generally indicated at 1. The pump 1 may comprise a housing 3 constructed so as to mount a cassette, generally indicated at 5, and a feeding set (broadly, a “pump set”), a fragmentary portion generally indicated at 7, removably received in the cassette. The feeding set 7 can comprise tubing indicated generally at 77 that provides a fluidic pathway between a source of nutritional liquid and a flushing liquid (FIG. 1). The left tubing 77 may be considered the upstream tube portion connected to a feed bag, while the right tubing 77 may be considered the upstream tube portion connected to a flush bag. Tubing 83 provide a fluidic pathway from the pump 1 to a user. In aspects of the disclosure the end user may be one of a patient or administrator of the enteral feeding pump. As will be explained in greater detail below, the pump 1 may comprise a flow monitoring system 6 (FIG. 4) that is capable of detecting and identifying a condition of the feeding set 7 loaded on the pump. More specially, for occlusion detection for thick formula feeds. As used herein, the term “load” means that the feeding set 7 is engaged with the pump 1 so that the feeding set is ready for operation with the pump to deliver fluid to the patient. As used herein, the term “conduit” means a tube portion.

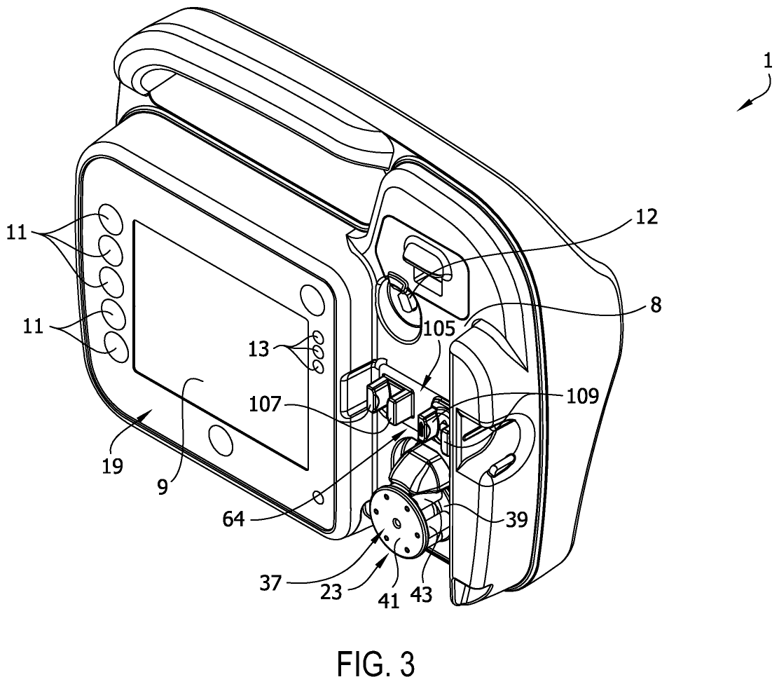

In the illustrated aspects, the cassette 5 is removably received in a cassette recess 8 in the housing 3 (FIG. 3). It will be appreciated that “housing” as used herein may include many forms of supporting structures (not shown), including without limitation multi-part structures and structures that do not enclose or house the working components of the pump 1. Moreover, various aspects and features of the present disclosure can be implemented without the recess 8. The pump 1 may also comprise a display screen 9 on the housing 3 that is capable of displaying information pertaining to the status and operation of the pump. One or more buttons 11 which can be proximate the display screen 9 can be provided for use in controlling and obtaining information from the pump 1, and one or more light emitting devices (“LEDs”) 13 can provide status information for the pump. In one aspect of the disclosure the light emitting devices may be any form of a device which emitted lights, for example, fiber optics, light emitting diodes, and the like. For example, the LEDs 13 may indicate proper or improper functionality of the pump 1. Additionally, for example, the LEDs may also indicate when fluid is properly or improperly flowing or not flowing through the feeding set 7. Legs (not shown) may be disposed at the bottom of the housing 3 to support the housing so that the display screen 9 is angled slightly upward for ease of viewing by a user.

The display screen 9 may be part of a front panel (generally indicated at 19) of the housing 3 and may be removably attached to the housing. The pump 1 may further include a pumping unit indicated generally at 23 comprising a pump motor 27 (FIG. 4) connected to a rotor shaft. A battery (not shown) may be received in the housing 3 for powering the pump motor. A power source other than or in addition to the battery could be used to energize the pump including one or more prime movers which drive the pumping unit through the rotor shaft. Another example of a pump with a rotor shaft is disclosed in U.S. Patent Publication No. 2020/0352827, the entire disclosure of which is herein incorporated by reference.

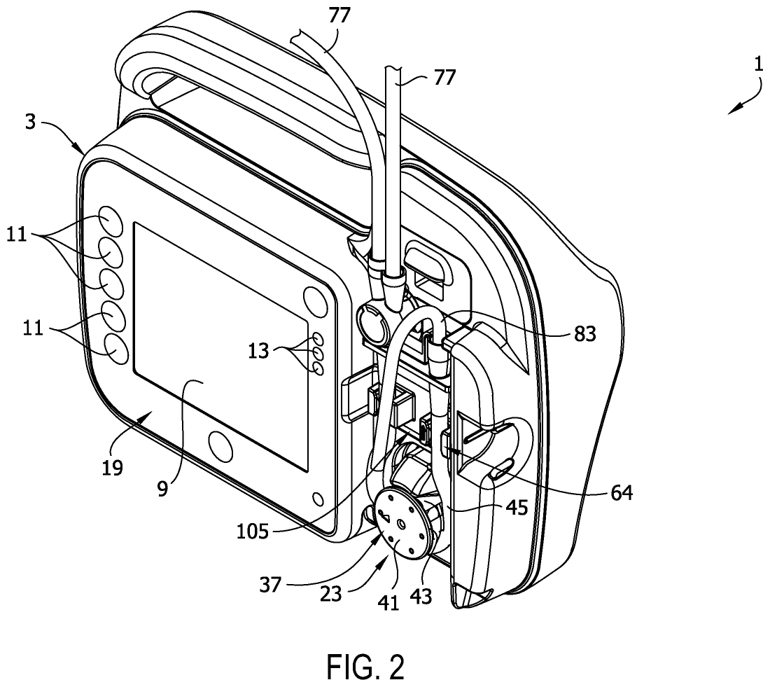

The pumping unit 23 can have a rotor (generally indicated at 37) which can be coupled to the rotor shaft. The rotor 37 may include an inner disk 39, an outer disk 41, and rollers 43 (preferably four, but only two of which are indicated). Inner disk 39 and outer disk 41 preferably lie in parallel planes, spaced from one another and rotatable about a shared axis. Rollers 43 are mounted between the inner disk 39 and the outer disk 41 for planetary rotation about the shared axis of the disks 39, 41. Each roller 43 is also mounted to the disks 39, 41 for rotation relative to the disks 39, 41 about its own longitudinal axis (FIGS. 2 and 3), which may be parallel to the shared axis of the disks 39, 41. As the rollers 43 rotate about the axis of the disks 39, 41, they engage a tube 45 (FIG. 2) of the feeding set 7 to deliver fluid through the feeding set, via peristaltic engagement, to a patient when the feeding set is received in the cassette 5 and the cassette is attached to the housing 3. Other numbers of rollers may also be envisioned and implemented. For example and without limitation, five or six rollers may also be used without departing from the scope of the disclosure.

The rollers 43 may engage the feeding set 7 for moving fluid through the feeding set. The delivery of fluid through the feeding set may be referenced below as providing aliquots of the thick formula feeds. In the illustrated aspect, the pump motor 27, rotor shaft, and rotor 37, may broadly be considered “a pumping device.” These components may be individually considered “a pumping device.” It will be understood that peristaltic pumps that use mechanisms other than rollers may fall within the scope of the present disclosure. However, other pumping devices (e.g., non-rotary devices) are envisioned.

As used herein, the portion of tubing 77 of the feeding set 7 leading to the rotor 37 is termed “upstream,” while the tubing 83 leading away from the rotor 37 to the patient is termed “downstream.” Rotation of the rotor 37 compresses the tube 45 of the feeding set 7 to drive fluid (e.g., a nutritional liquid) in a patient direction from the upstream to the downstream side of the feeding set. Although an example feeding set 7 is shown, feeding sets of other configurations and other types of pump sets (not shown) can be used.

Referring to FIG. 2-4, the monitoring system 6 (FIG. 4) is capable of detecting and identifying a condition of the feeding set 7 loaded on the apparatus. For example, a condition of the feeding set 7 may relate to the flow of liquid through the set, whether the set is mounted properly on the pump, whether there is an occlusion, or other circumstances pertaining to the feeding set or its operation. For example, the flow of liquid through the set may include the lack of or improper flow of liquid through the set. Additionally, for example, if the set is improperly mounted on the pump, fluid may not flow properly through the set. Additionally, for example, when the set is improperly mounted on the pump an occlusion may be present within the tubing.

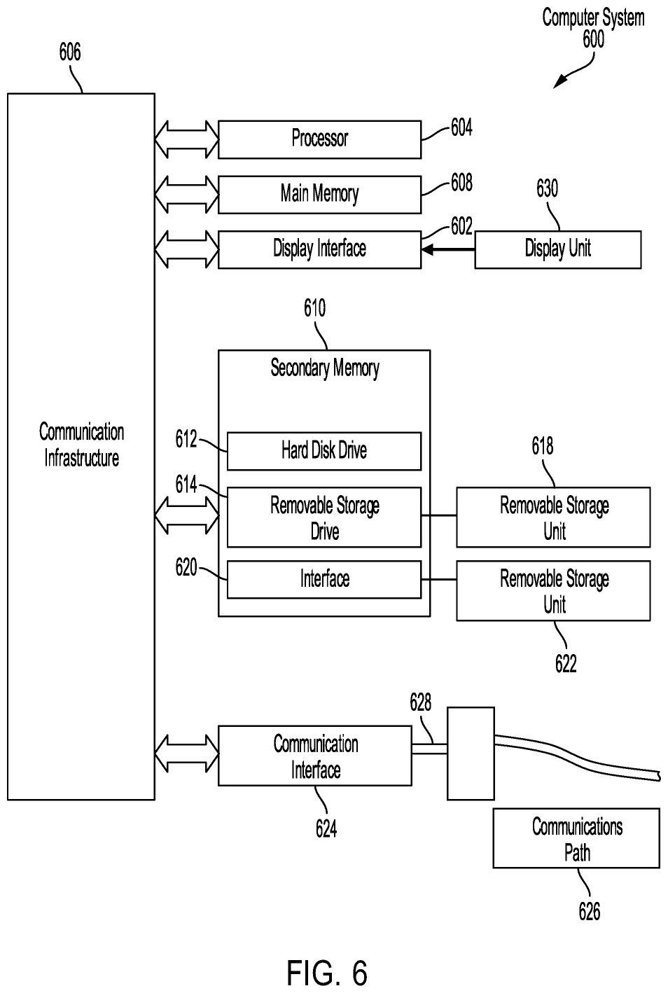

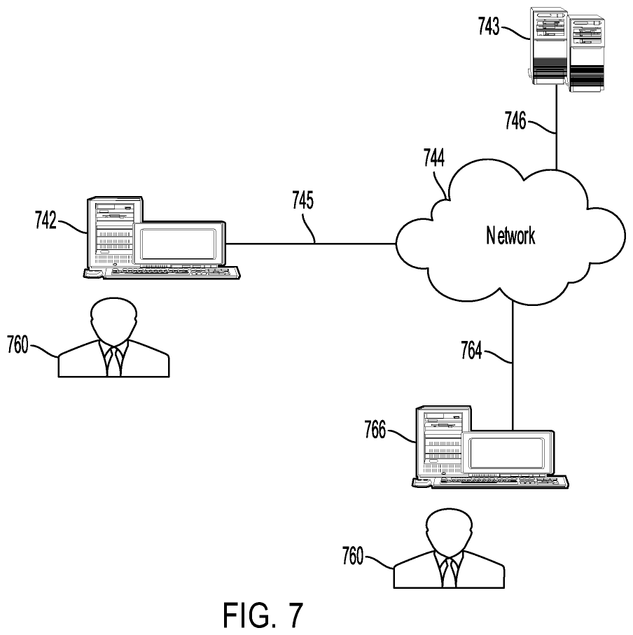

The pump 1 may further comprise a microprocessor 62 in communication association with a sensor 64. The microprocessor 62 may control and manage the operation of the various components of the pump 1. A software subsystem 66 may be operatively associated with the microprocessor 62 and operatively associated with the monitoring system 6 to provide a means for the pump 1 to detect and identify a condition of the feeding set 7. It is to be understood that in the described aspect, the flow monitoring system 6, the software subsystem 66, pump electronics 68, the microprocessor 62 and memory 70 may be broadly considered a “control circuit.” These components may be individually considered a “control circuit.” Moreover, other types of control circuits may be used within the scope of the present disclosure. As described in reference to FIGS. 6 and 7 below, the control circuit may be implemented in relation to the various components. The switch 72 may include a plurality of switches, for example, a start button, as described below.

The sensor 64 may comprise one or more ultrasonic sensors and or pressure sensors. The sensor 64 may be located on the housing 3 of the pump 1 and positioned to detect the presence of fluid as well as one or more properties of a fluid in the feeding set 7, e.g., an occlusion of the fluid in the feeding set. In the illustrated aspect, the sensor 64 is positioned in recess 8 and is adapted to securely receive a portion of the tube 45 therein when the feeding set 7 is loaded on the pump 1. In order for the sensor 64 to detect the presence of fluid in the tube 45 of the feeding set 7, the tube may be engaged and retained within a sensor track 105 (FIG. 3) configured to receive an upstream portion and a downstream portion of the tube 45. Once the tube 45 is engaged within the sensor track 105 and the remaining portions of the feeding set 7 are engaged with the pump 1, the monitoring system 6 may become operational. For example, the monitoring system 6 may become operationally functional when a positive engagement of the tube 45 within the sensor track 105 has been identified by the receipt of an acceptable signal, e.g., an ultrasonic signal, by one or more detectors or receivers. The sensor 64 may be positioned perpendicular to the direction of the feeding set 7. For example the sensor 64 may be positioned to read horizontally, while the feeding set 7 may be positioned to flow fluid vertically.

In one aspect of the disclosure, the sensor 64 may comprise a first sensor component 107, 109 for transmitting an ultrasonic signal through an upstream and downstream portion of the tube 45, respectively, and a second sensor component 107, 109 configured to receive and detect the ultrasonic signal emitted from the first sensor component. Upon receipt of the ultrasonic signal from the first sensor component 107, 109, the second sensor component 107, 109 may detect the presence of fluid within the tube 45 based on the characteristics of the ultrasonic signal received by the second sensor component and communicated to the microprocessor 62. The first and second sensor components 107, 109 may each comprise identical or substantially identical sensor configurations. For example, each sensor component 107, 109 may comprise ultrasonic crystals whereby each sensor component can be operated as a transmitter for transmitting the ultrasonic signal, or as a detector for detecting the ultrasonic signal depending on the way in which the components are energized. Therefore, the direction of the ultrasonic signal is not confined to a single direction between the sensor components 107, 109 but instead can be directed in both directions between the sensor components. In another aspect of the disclosure, the sensor 64 may be pressure or force sensor.

The sensor 64 may detect the presence or absence of fluid in the tubing to give a basic indication of the operational status of the pump 1. The ultrasonic signal emitted from the sensor components 107, 109 may be responsive to the presence of fluid in the tube 45 such that fluid in the tube will produce an increase in amplitude of the signal as compared to a signal where fluid is not in the tubing. As such, an ultrasonic signal passing through an all air media will not produce a signal at the detector. Based on the characteristics of the received ultrasonic signal communicated to the microprocessor 62, the software subsystem 66 may determine whether fluid is present within the feeding set 7. Other types of sensors for measuring one or more fluid properties or characteristics, including viscosity, other than ultrasonic sensors can be used. The flow monitoring system 6 may also detect other conditions of the feeding set 7, the fluid within the feeding set, and the fluid coupled with the feeding set without departing from the scope of the disclosure.

In one aspect of the disclosure, the pump 1, described above, may be configured to operate with thickened enteral formula and blenderized tube feeds (“BTF”). Thickened enteral formula and BTF are administered to prevent or reduce enteral nutrition related complications and intolerance symptoms, such as nausea, vomiting, and gastroesophageal reflux. However, increased force is needed to push high viscosity feeds through connectors and tubing which often causes false occlusion alarms when given via enteral feeding pump. Additionally, administration of blenderized tube feeds can be complicated by bits of solid food that may obstruct the feeding tube.

The International Dysphagia Diet Standardization Initiative (IDDSI) is a global standard with terminology and definitions to describe texture modified foods and thickened liquids used for individuals. The IDDSI framework consists of a continuum of 8 levels (0-7). The levels, for example, are as follows: 7 are regular foods; 6 is soft and bite-sized foods; 6 is minced and moist foods; 4 is pureed foods/extremely thick; 3 is liquidized foods/moderately thick; 2 is mildly thick food; 1 is slightly thick foods; and 0 is thin foods. Further, the IDDSI framework has two distinct portions, solid foods and liquids. Specifically, solid foods are classified from levels 3-7, and liquids are classified from levels 0-4. Thus, levels 3 and 4 overlap between solid foods and liquids. In one aspect of the disclosure, the fluids being referenced as high viscosity feeds, thickened enteral formula or use with blenderized tube feeds are rated between a level of 2-4 level on the IDDSI framework. In another aspect of the disclosure, the fluids being referenced as high viscosity feeds, thickened enteral formula or use with blenderized tube feeds also include slightly thick food, thin foods and liquids which are rated between a level of 0-4 level on the IDDSI framework.

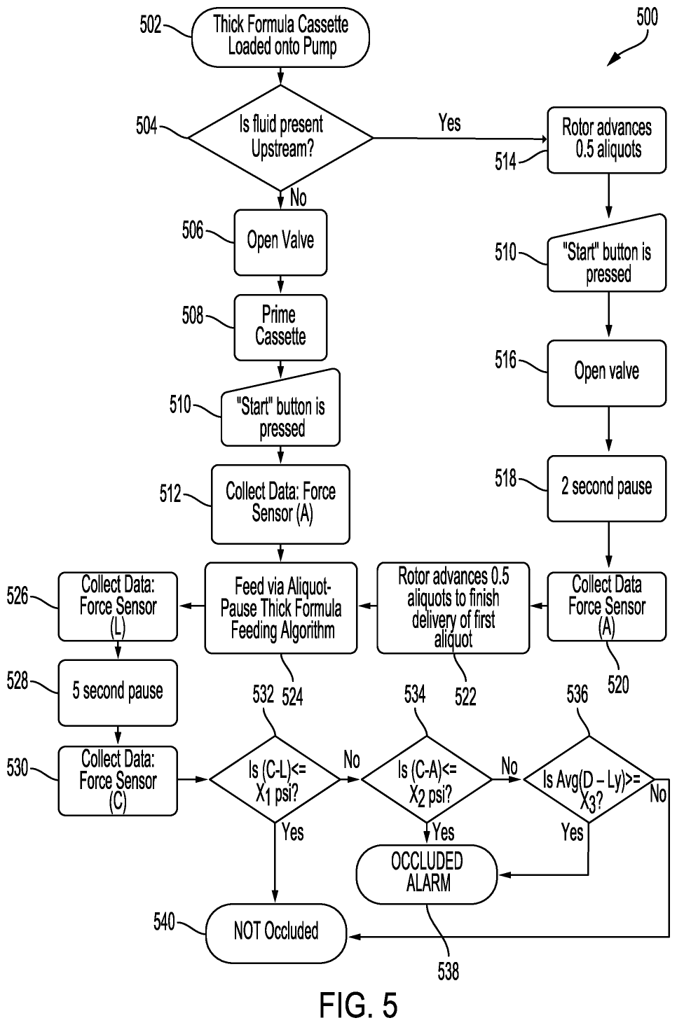

Turning to FIG. 5, therein shown is a flowchart of an example method 500 of an occlusion detection routine in accordance with an implementation of the present disclosure. Specifically, FIG. 5 is directed to an occlusion detection routine for use with high viscosity feeds and the pump 1 as illustrated in FIGS. 1-4 and described above. Most occlusion detection algorithms with pumps use a force sensor to detect increases in pressure within the tubing which can indicate a blockage restricting the flow of enteral nutrition. Due to their high viscosity, normal pumping of thickened enteral formula and blenderized tube feeds often exceed the force thresholds that might otherwise indicate that a feeding set is occluded. Typically, blenderized tube feeds are administered via syringe or gravity feed to avoid the use of an enteral feeding pump which causes false occlusion alarms. Nurses and/or caregivers may thin blenderized tube feeds with 2-8 oz of fluid prior to administration via the feeding pump. The addition of this liquid can exceed a tolerable volume of food for a patient, and restrict the ability of a patient to receive adequate calories. Further, pumps and/or feeding sets are sometimes modified by the nurses and/or caregivers to override safety features to turn off occlusion detection and avoid false alarms.

In view of these known issues described above, in order to distinguish between a thin, commercial formula that builds pressure when an occlusion is present, and a thick formula pumping normally without occlusion, the method 500 measures the pressure decay at the end of a feeding cycle. A pressure decay equal to or exceeding about 4 pounds per square inch (psi), for example, may indicate that the feeding set is not occluded. In another example, a pressure decay equal to or exceeding about 3 psi, for example, may indicate that the feeding set is not occluded. These thresholds of 3 and 4 psi are examples and are dependent upon the rate of the fluid flow, for example mL/hr. If this decay at the end of the feeding cycle does not occur, the relative pressure change of the feeding set is assessed. A relative pressure increase of −5 psi during the feed cycle may indicate an occlusion. If a feeding set is installed into the pump 1 with an existing occlusion, the pump 1 will not sense a notable pressure decay at the end of a feeding cycle, and may not sense a notable pressure increase during the feed. In this example, the force values associated with the last 12 aliquots are assessed for erratic behavior. Although 12 aliquots are an example, any number of aliquots may be averaged. In an occluded feeding set, the average change in magnitude of the force readings taken after the delivery of each aliquot is >=3 PSI. In an un-occluded feeding set, the force readings taken aliquot to aliquot are much more stable. In addition to these fundamental steps, when fluid has been detected in a thick formula set upon installation, the rotor advances 0.5 aliquot prior to the cassette valve opening. When the valve is opened at the beginning of a feed, any residual pressure is released and the feeding set is returned to an unpressurized state. This returns a previously used set to baseline conditions and allows pressure changes to be easily assessed. Note, the thresholds listed above are for example purposes, and do not take away from the disclosure as a whole, as the thresholds may be set based upon different factors, for example, feed rate, tube size, and/or IDDSI level, etc.

As known in the art, an aliquot is an amount that is an exact divisor of the whole quantity of a substance. A feed cycle of X aliquots is determined by time. For example, 1 feed cycle is 1 minute of feed time. Further, as noted above, the PSI values/thresholds listed above are converted from force readings using a calibration constant from the pump.

Turning to block 502 of method 500 for occlusion detection for thick formula feeds, a thick formula cassette is loaded into or onto the pump 1 by a nurse and/or caregiver. At block 504 the system determines if fluid is present upstream. The pump 1 may be configured to run a fluid detection routine whereby an ultrasonic sensor is operated to emit an ultrasonic signal through a portion of the tubing to determine a condition of the feeding set. If the sensor reading is above a predetermined threshold, the pump provides an initial indication that fluid is present in the tubing. If the sensor reading is at or below the predetermined threshold, the pump provides an indication that no fluid is present in the tubing.

If the system determines that fluid is not present within the system, the system opens the valve at block 506. The valve 12, as described in reference to FIGS. 1-3, is the location of where the upstream tubing enters the pump 1. The valve 12 closes/opens the lumen of the tubing located in the feeding set and is a component of the cassette. The physically closing/opening is performed by turning a mechanical mechanism on the pump which mates with the cassette when installed in the pump 1. At block 508 the system primes the cassette. The system may prime the cassette by filling the feeding tube with a portion of the blenderized tube feeds. At block 510, the “Start” button or an activation button on the pump 1 is pressed by a user. At block 512 the system collects data from a force sensor, as described above. The data collected is referenced as “A Value.” The A Value is the force reading taken at the beginning of the first cycle of the feed cycle before pumping starts by the system in a normal aliquot-pause feeding algorithm. An example of a normal aliquot-pause feeding algorithm is disclosed in U.S. patent application Ser. No. 17/750,041, the entire disclosure of which is herein incorporated by reference. Further, note the force values are taken as PSI.

Turning back to block 504, if the system determines that fluid is present upstream, the system moves to block 514. At block 514 the rotor advances 0.5 aliquots. At block 510, the “Start” button or an activation button on the pump 1 is pressed by the user. At block 516, the system opens the valve. At block 518 the system pauses, for example, 2 seconds but may be as between 1-3 seconds. During the 2 second pause, the pressure in the system is stabilizing. At block 520 the system collects data from a force sensor, as described above, as A Value. At block 522 the rotor again advances 0.5 aliquots to finish delivery of the first aliquot of the thickened enteral formula.

At block 524 the system steps converge to perform the same steps regardless of the presence or the lack of presence of fluid upstream. At block 524, the system performs feeding via an aliquot-pause thick formula feeding algorithm. As discussed above, an example of an aliquot-pause feeding algorithm is disclosed in U.S. patent application Ser. No. 17/750,041, the entire disclosure of which is herein incorporated by reference. At block 536 the system collects data from the force sensor, as described above. The data collected is referenced as “L Value.” The L Value is the force reading taken after the final aliquot of the cycle has been deliver, but prior to a deliberate pause in the system. At block 528, the deliberate pause, for example, being 5 seconds. In another aspect of the disclosure the deliberate pause may be between 2-7 seconds.

At block 530 the system collects data from the force sensor, as described above. The data collected is referenced as “C Value.” The C Value is a force reading taken after a deliberate pause at the end of a feeding cycle. For example, the C Value is taken after the 5 second pause in the system at block 528.

At block 532, the system subtracts the L Value from the C Value, and determines if the result is less than or equal to a first threshold in PSI (X₁). The first threshold may be between −10 and 2 PSI, more specifically between −6 and −2 PSI. and more specifically −4 PSI. If the comparison is yes, the system is determined to be not occluded at block 540. If the result is greater than the first threshold, the system moves to block 534.

At block 534 the system subtracts the A Value from the C Value, and determines if the result is greater than or equal to a second threshold in PSI (X₂). The second threshold may be between 0 and 10 PSI, more specifically between 4 and 8 PSI, and more specifically 5 PSI. If the comparison is yes, the system is determined to be occluded. At block 538 the system triggers an alarm to notify the nurse/caregiver of the occlusion. In one aspect of the disclosure, the alarm may be visual, auditory or a combination thereof. The visual alarm may be provided to a user by the display screen 9 (FIG. 1) and or the LEDs 13, and the auditory alarm may be provided to a user by a speaker. The alarm may be provided to a user to correct, fix or adjust issues with the system. If the result is less than the second threshold, the system moves to block 536.

At block 536 the system determines if the average of Δ_{Ly} is greater than or equal to third threshold in PSI (X₃). The third threshold may be between −6 and 8 PSI, more specifically between −2 and 5 PSI, and more specifically 3 PSI. Where Δ_{Ly} is the average change in magnitude of force readings taken after every aliquot-pause for the last two full rotor revolutions. In one aspect of the disclosure, this would be a total of 12 force readings. Note, this value may not include C values. Further, if the feed rate is less than are 21 mL/hour, than only 11 force readings may be implemented for this calculation. If the comparison is yes, the system is determined to be occluded. At block 538 the system triggers an alarm to notify the nurse/caregiver of the occlusion. If the comparison is no, the system is determined to be not occluded at block 540.

Further, block 536 may also be independently or dependently implemented to a downstream occlusion algorithm when the pump 1 is running in bolus max mode. Bolus max mode is when the pump 1 is operating at a max rate of feeding (for example, 800 mL//hr). Bolus max mode is available when a standard feed cassette (non-thick formula) is loaded in to the pump 1. When running on bolus max mode, the pump 1 may operate at 6 minute cycles. Thus, portions of the method and/or system, for example block 536, may be implemented with any pump 1.

Aspects of the present disclosure may be described in the general context of computer-executable instructions, such as program modules, executed by one or more computers or other devices. The computer-executable instructions may be organized into one or more computer-executable components or modules including, but not limited to, routines, programs, objects, components, and data structures that perform particular tasks or implement particular abstract data types. Aspects of the disclosure may be implemented with any number and organization of such components or modules. For example, aspects of the disclosure are not limited to the specific computer-executable instructions or the specific components or modules illustrated in the figures and described herein. Other aspects of the disclosure may include different computer-executable instructions or components having more or less functionality than illustrated and described.

Further, the order of execution or performance of the operations in aspects of the disclosure illustrated and described herein are not essential, unless otherwise specified. That is, the operations may be performed in any order, unless otherwise specified, and aspects of the disclosure may include additional or fewer operations than those disclosed herein. For example, it is contemplated that executing or performing a particular operation before, contemporaneously with, or after another operation is within the scope of aspects of the disclosure.

In operation, the microprocessor 62 executes computer-executable instructions such as those illustrated in the figures to implement aspects of the disclosure. Aspects of the disclosure may also be practiced in distributed computing environments where tasks are performed by remote processing devices linked through a communications network. In a distributed computing environment, program modules may be located in both local and remote computer storage media including memory storage devices.

Aspects of the present disclosure may be implemented using hardware, software, or a combination thereof and may be implemented in one or more computer systems or other processing systems. In an aspect of the present disclosure, features are directed toward one or more computer systems capable of carrying out the functionality described herein. An example of such a computer system 600 is shown in FIG. 6.

Computer system 600 includes one or more processors, such as processor 604. The processor 604 is connected to a communication infrastructure 606 (e.g., a communications bus, cross-over bar, or network). Various software implementations are described in terms of this example computer system. After reading this description, it will become apparent to a person skilled in the relevant art(s) how to implement implementations of the disclosure using other computer systems and/or architectures.

Computer system 400 may include a display interface 602 that forwards graphics, text, and other data from the communication infrastructure 606 (or from a frame buffer not shown) for display on a display unit 630. Computer system 600 also includes a main memory 608, preferably random access memory (RAM), and may also include a secondary memory 610. The secondary memory 610 may include, for example, a hard disk drive 612, and/or a removable storage drive 614, representing a floppy disk drive, a magnetic tape drive, an optical disk drive, a universal serial bus (USB) flash drive, etc. The removable storage drive 614 reads from and/or writes to a removable storage unit 618 in a well-known manner. Removable storage unit 618 represents a floppy disk, magnetic tape, optical disk, USB flash drive etc., which is read by and written to removable storage drive 614. As will be appreciated, the removable storage unit 618 includes a computer usable storage medium having stored therein computer software and/or data.

Alternative implementations of the present disclosure may include secondary memory 610 and may include other similar devices for allowing computer programs or other instructions to be loaded into computer system 600. Such devices may include, for example, a removable storage unit 622 and an interface 620. Examples of such may include a program cartridge and cartridge interface (such as that found in video game devices), a removable memory chip (such as an erasable programmable read only memory (EPROM), or programmable read only memory (PROM)) and associated socket, and other removable storage units 622 and interfaces 620, which allow software and data to be transferred from the removable storage unit 622 to computer system 600.

Computer system 600 may also include a communications interface 624. Communications interface 624 allows software and data to be transferred between computer system 600 and external devices. Examples of communications interface 624 may include a modem, a network interface (such as an Ethernet card), a communications port, a Personal Computer Memory Card International Association (PCMCIA) slot and card, etc. Software and data transferred via communications interface 624 are in the form of signals 628, which may be electronic, electromagnetic, optical or other signals capable of being received by communications interface 624. These signals 628 are provided to communications interface 624 via a communications path (e.g., channel) 626. This path 626 carries signals 628 and may be implemented using wire or cable, fiber optics, a telephone line, a cellular link, a radio frequency (RF) link and/or other communications channels. In this document, the terms “computer program medium” and “computer usable medium” are used to refer generally to media such as a removable storage unit 618, a hard disk installed in hard disk drive 612, and signals 628. These computer program products provide software to the computer system 600. Implementations of the present disclosure are directed to such computer program products.

Computer programs (also referred to as computer control logic) are stored in main memory 608 and/or secondary memory 610. Computer programs may also be received via communications interface 624. Such computer programs, when executed, enable the computer system 600 to perform the features in accordance with implementations of the present disclosure, as discussed herein. In particular, the computer programs, when executed, enable the processor 604 to perform the features in accordance with implementations of the present disclosure. Accordingly, such computer programs represent controllers of the computer system 600.

In an aspect where the disclosure is implemented using software, the software may be stored in a computer program product and loaded into computer system 600 using removable storage drive 614, hard drive 612, or communications interface 620. The control logic (software), when executed by the processor 604, causes the processor 604 to perform the functions described herein. In another aspect of the present disclosure, the system is implemented primarily in hardware using, for example, hardware components, such as application specific integrated circuits (ASICs). Implementation of the hardware state machine so as to perform the functions described herein will be apparent to persons skilled in the relevant art(s).

FIG. 7 is a block diagram of various example system components, in accordance with aspects of the present disclosure. FIG. 7 shows a communication system 700 including one or more accessors 760 (also referred to interchangeably herein as one or more “users”), one or more terminals 742. Terminals 742 can include system 100 and or 200, described above, or a related system, and/or the like. In one aspect, data for use in accordance with aspects described herein may be input and/or accessed by accessors 760 via terminal 742, such as personal computers (PCs), minicomputers, mainframe computers, microcomputers, telephonic devices, or wired/wireless devices, such as personal digital assistants (“PDAs”) and RFID readers (e.g., handheld, mobile, cabinets, etc.) coupled to a server 743, such as a PC, minicomputer, mainframe computer, microcomputer, or other device having a processor and a repository for data and/or connection to a repository for data, via, a network 744 for instance, such as the Internet or an intranet, and couplings 745, 746, 764. The couplings 745, 746, 764 may include wired, wireless, or fiber-optic links. In another example variation, the method and system in accordance with aspects described herein operate in a stand-alone environment, such as on a single terminal.

The aspects discussed herein can also be described and implemented in the context of computer-readable storage medium storing computer-executable instructions. Computer-readable storage media includes computer storage media and communication media, and may be, flash memory drives, digital versatile discs (DVDs), compact discs (CDs), floppy disks, and tape cassettes. Computer-readable storage media can include volatile and nonvolatile, removable and non-removable media implemented in any method or technology for storage of information such as computer readable instructions, data structures, modules or other data.

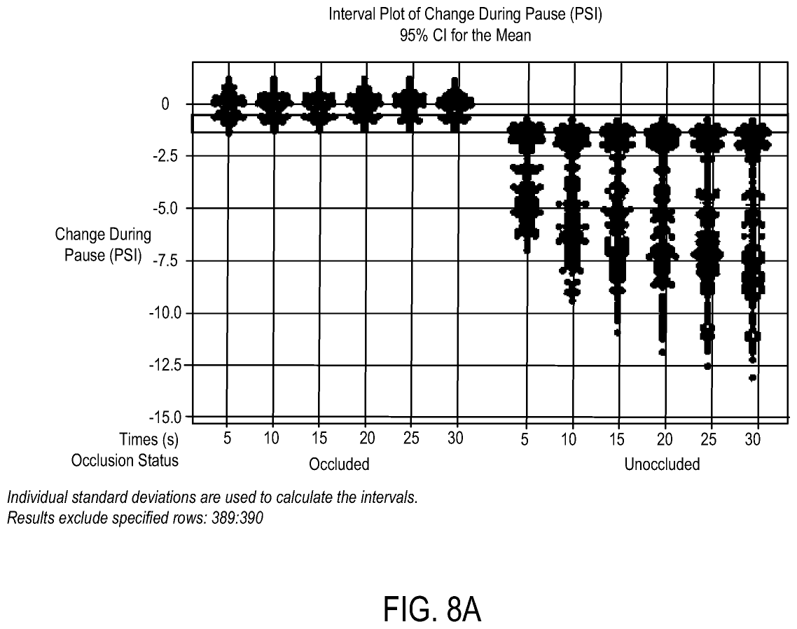

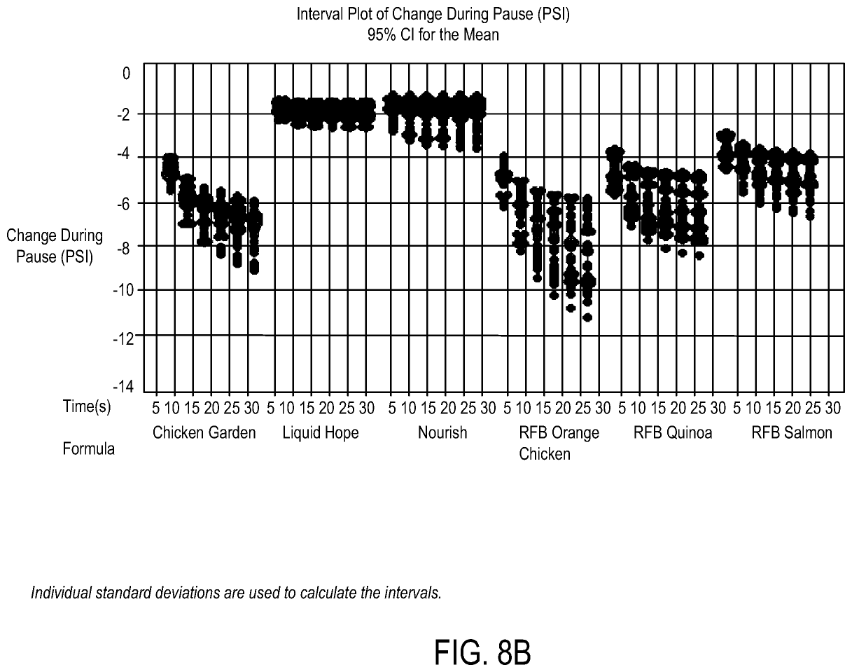

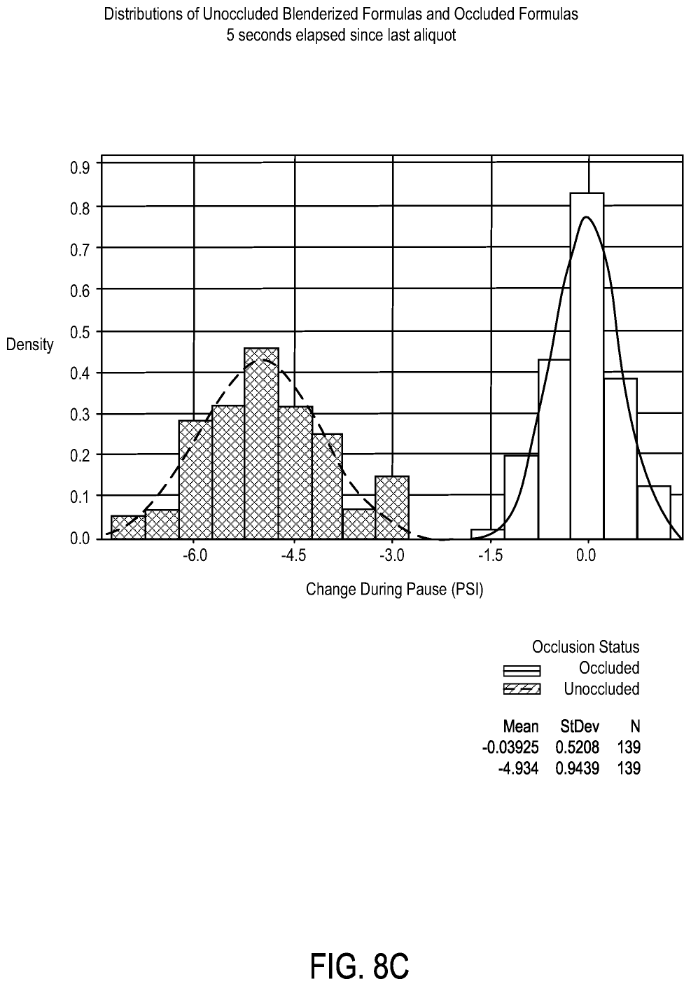

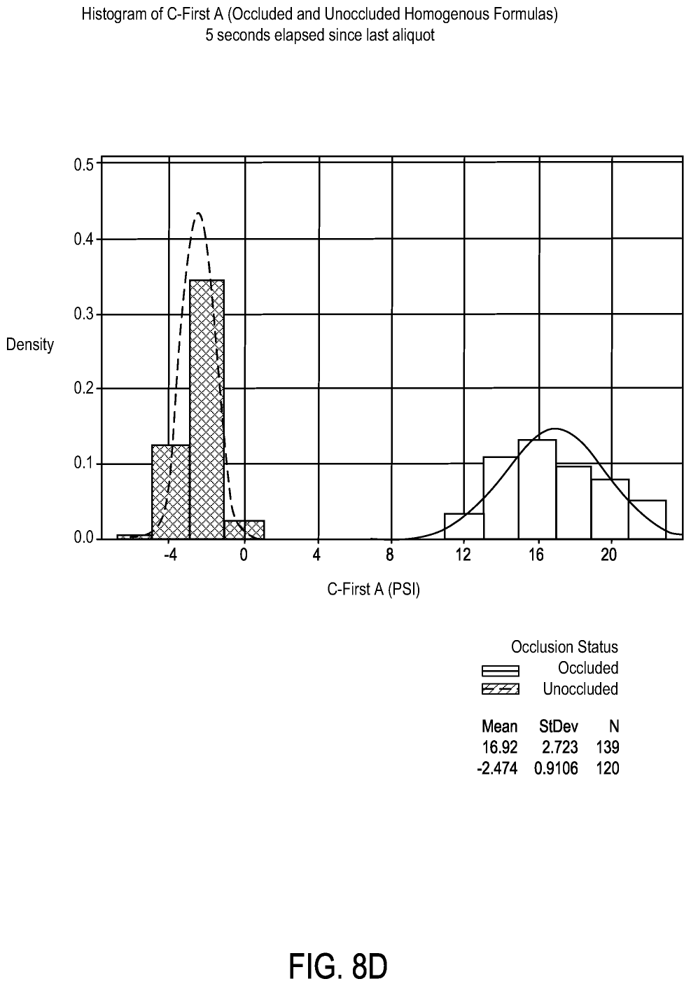

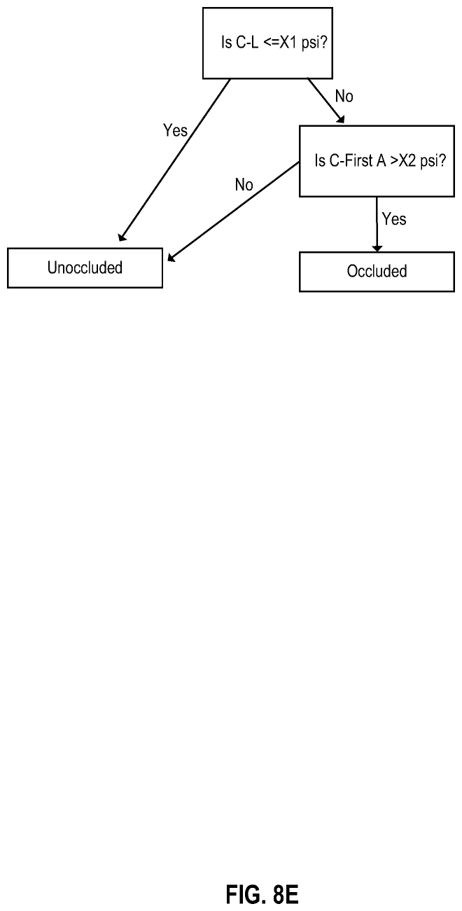

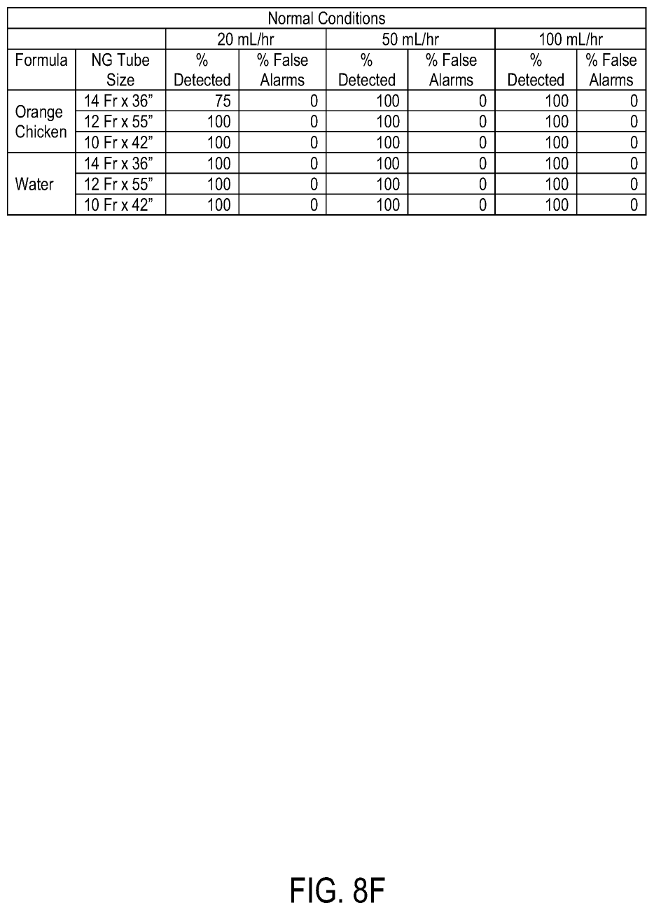

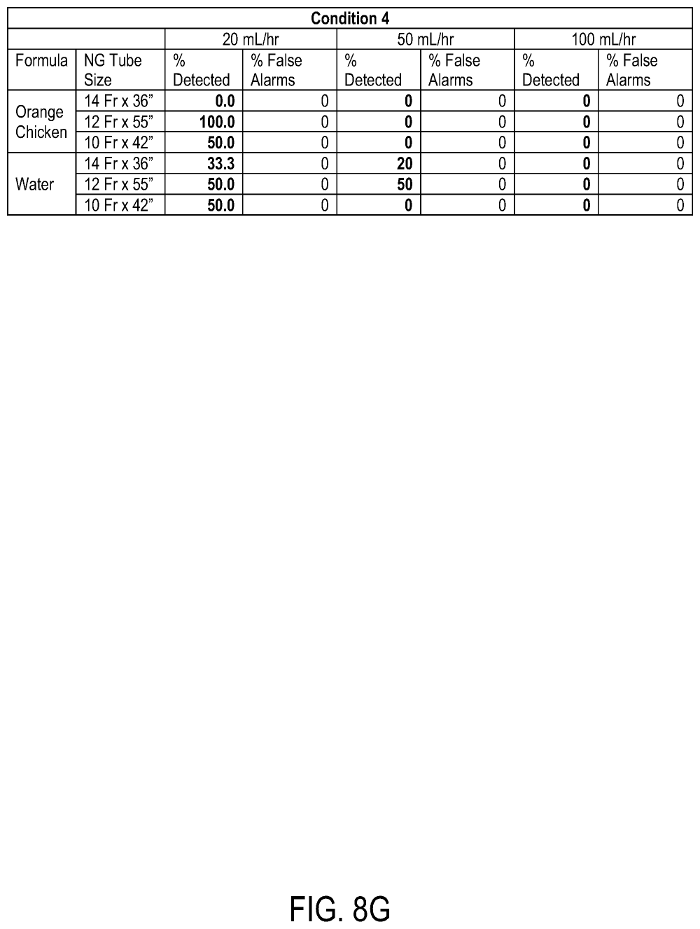

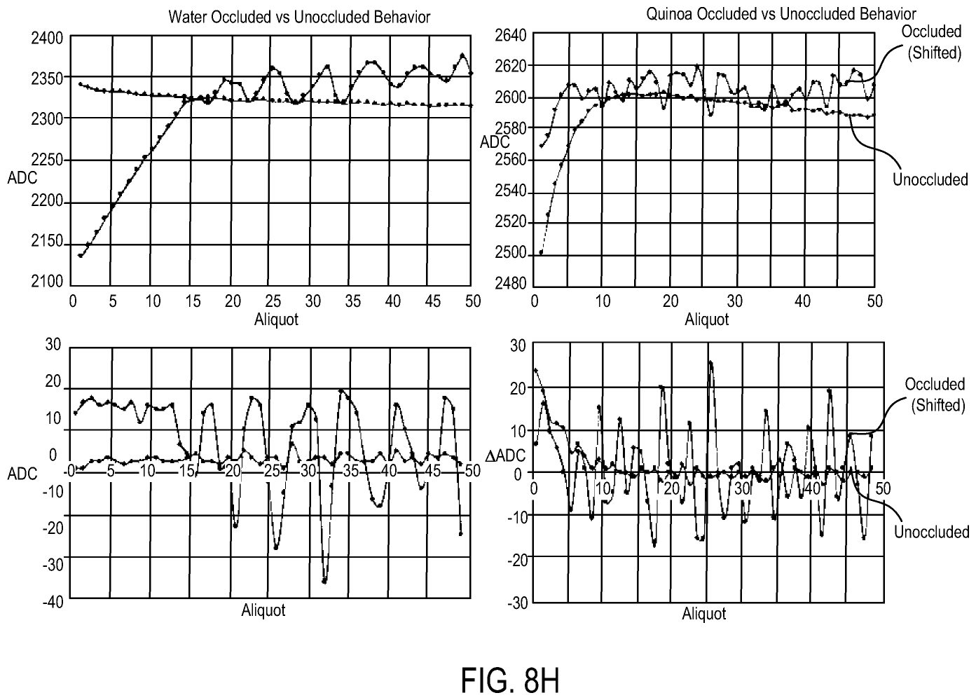

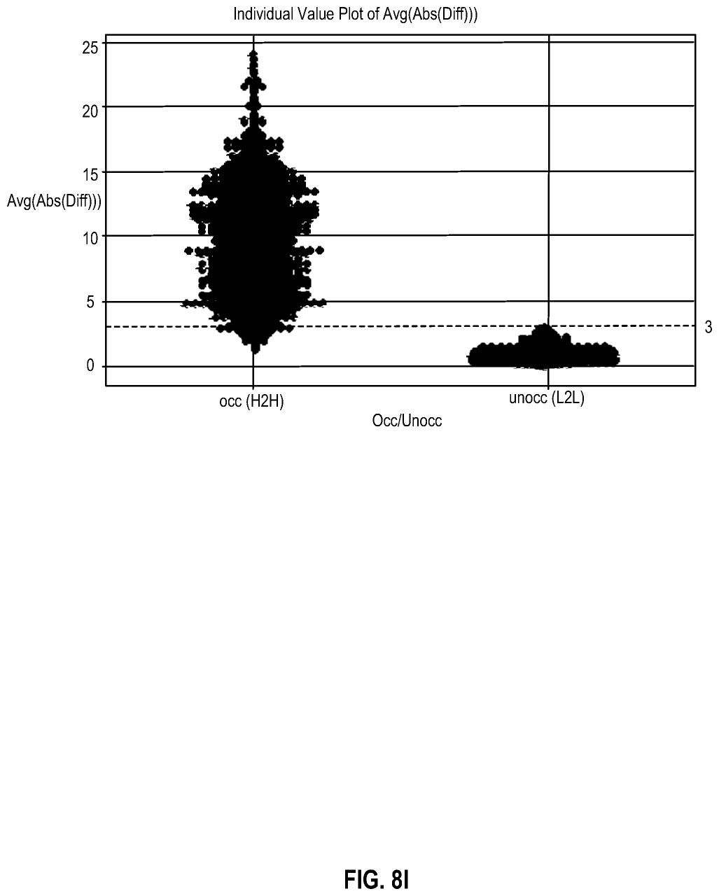

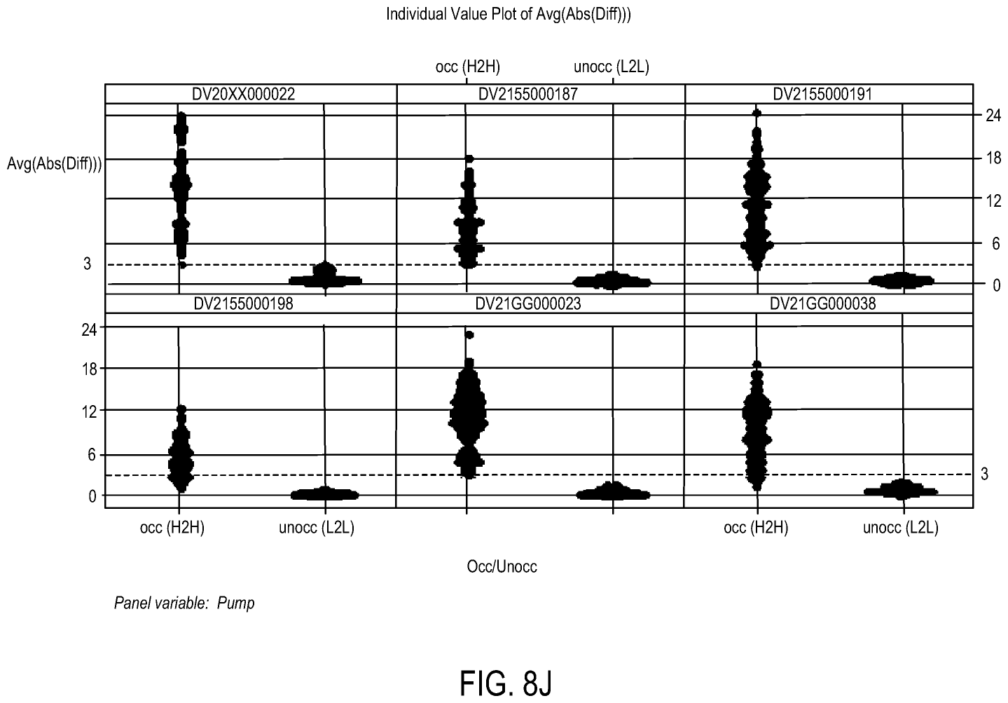

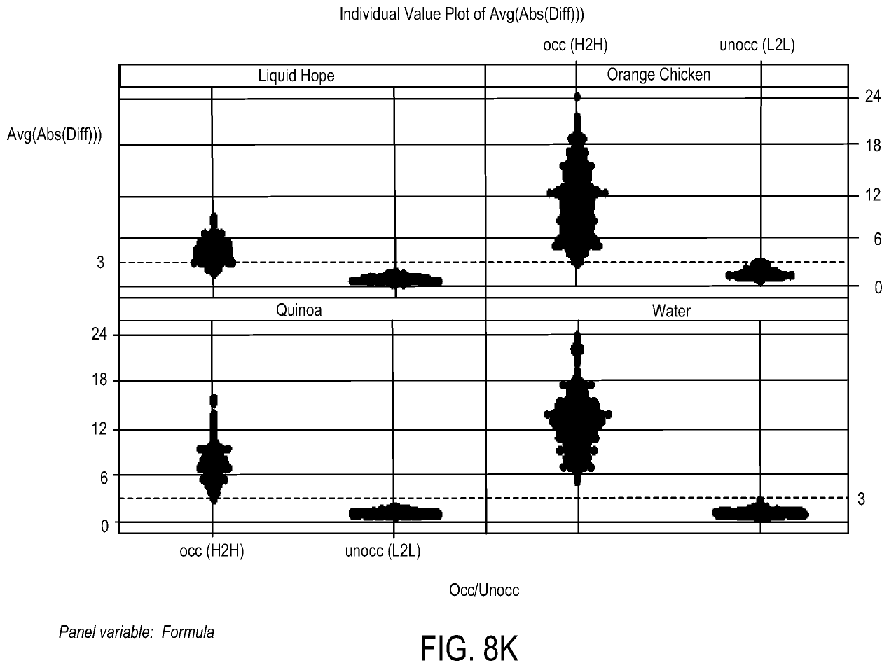

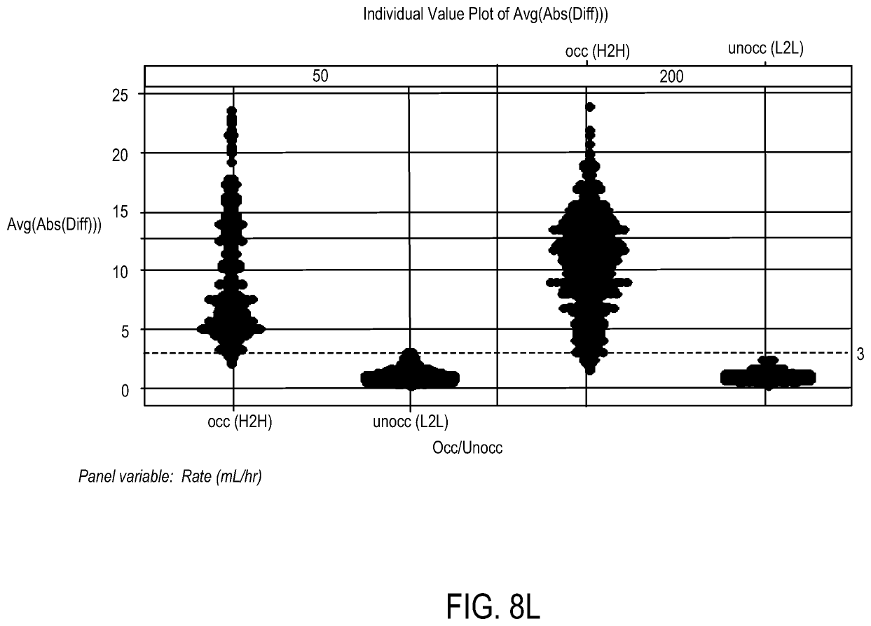

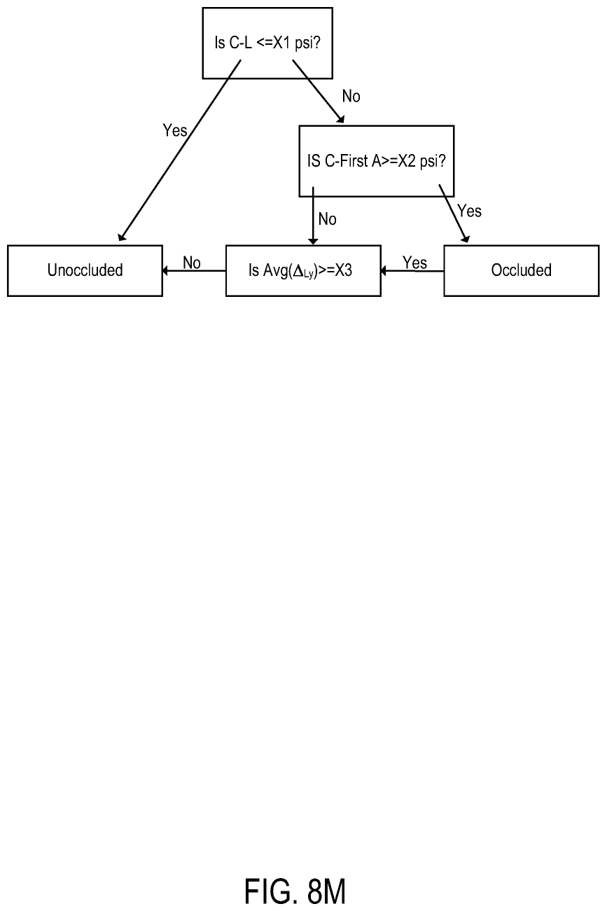

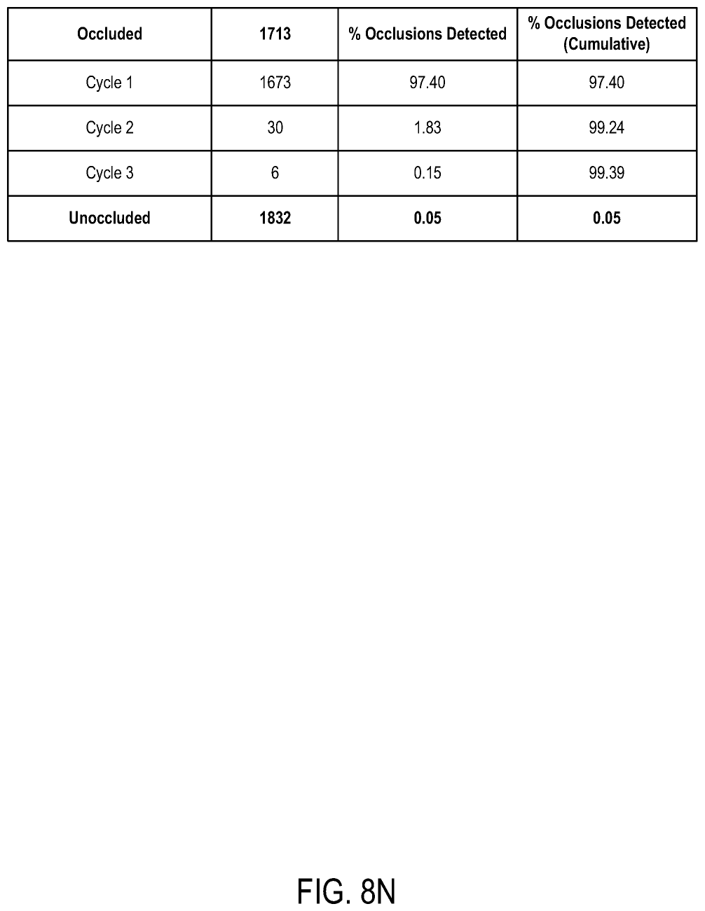

Referring to FIGS. 8A-8N, therein illustrated are example results associated with the occlusion detection routine in accordance with an implementation of the present disclosure. FIGS. 8A-8N are exemplary results which demonstrate the steps taken to determine the thresholds determined and implemented in blocks of 500 of FIG. 5.

Referring to FIG. 8A, therein illustrated is an interval plot change during the pause of the pump, as described above. Specially, the graph illustrates behavior differences between occluded and unoccluded feeding sets, with a variety of blenderized/thick formulas, IDDSI levels 2-4 over time in a period of pump inactivity. The graph illustrates the change in pressure (psi) from a time zero (L), when the last aliquot was delivered in a given feeding cycle, to the elapsed time shown on the x axis. The means demonstrates that unoccluded feed sets provide for a drop in pressure during a period of inactivity that is not observed in occluded sets. Note, there is some overlap in the distributions which is highlighted.

Referring to FIG. 8B, therein illustrated is a second interval plot of change during the pause of the pump, as described above. This graph illustrates the unoccluded feeding sets from the graph in FIG. 8A but separated by formula type. Specially, this graph illustrates that the blenderized, and generally thicker formulas (IDDSI level 4 for example), demonstrate a more significant drop in pressure during a time of inactivity of the pump, compared to the more homogenous, and generally thinner, formulas (IDDSI level 2 for example).

Referring to FIG. 8C, therein illustrated are distributions of unoccluded blenderized formulas and occluded formulas. Specifically, the graph illustrates a histogram of the pressure change during a 5 second period of rest (pump inactivity) from the time the last aliquot was delivered in a feeding cycle comparing occluded feedings sets (all formula types), as compared to the unoccluded blenderized formulas. The graph demonstrates that there is a separation in the distributions which allows a threshold to be established. Specially, this graph provides the basis for how the first part of the thick formula occlusion detection algorithm was derived: (C−L)<=−4 psi (as described above with reference to block 532 of FIG. 5), where the L Value is the force reading taken after the last aliquot of the feeding cycle has been delivered and C Value is the force reading taken after a deliberate 5 second pause. As described above, if this criterion is met, the system can determine that the feeding set is unoccluded.

Referring to FIG. 8D, therein illustrated is a histogram of the C Value minus the A Value of both the occluded and unoccluded homogenous formulas. This graph illustrates situations in which there is not a pressure drop during a 5 second pause after the last aliquot is delivered in a feeding cycle. Specifically, this graph demonstrates that there is a pressure increase evident only in occluded sets, as measured from the beginning of the first feed cycle prior to pumping (Value A) to 5 seconds post pump activity in a given feeding cycle (Value C). Moreover, is a separation in the distributions which allows a threshold to be established. Specifically, this graph provides the basis for how the second criterion for the thick formula occlusion detection algorithm was derived: (C−A)>=5 psi (as described above with reference to block 534 of FIG. 5).

Referring to FIG. 8E, therein generally illustrated is a flow chart/algorithm determined based on the data interpreted by FIGS. 8A-8D. The accuracy of the algorithm of FIG. 8E was tested using a thick, blenderized formula (IDDSI 4) and water (IDDSI 0) using a variety of nasogastric tub sizes at several feeding rates. The results of this testing for normal conditions are illustrated in FIG. 8F, described below. Normal conditions indicate that an unoccluded feeding set was loaded onto a pump, the feed set was then primed, the feed was initiated without issues, and an occlusion was simulated during normal feeding by clamping the feeding set. The conditions shown in FIG. 8F were sufficient to detect occlusions during these use conditions.

The accuracy of the algorithm of FIG. 8E was tested using a thick, blenderized formula (IDDSI 4) and water (IDDSI 0) using a variety of nasogastric tub sizes at several feeding rates. The results of this testing for non-normal conditions are illustrated in FIG. 8G. A non-normal condition occurs when an occluded set is loaded onto a pump prior to a feed being initiated. The criteria shown in FIG. 8G was not sufficient to detect occlusions during this use conditions.

Referring to FIG. 8H, when observing force readings (as measured with an analog to digital conversion, ADC, for example a digital value proportional to an analog measurement) over time, as illustrated in chronological sequences by aliquot, there were more erratic and noisy pressure profiles evident in feeding sets that were occluded than feeding sets that were unoccluded. This erratic behavior was quantified by taking the average change in magnitude from aliquot to aliquot for the last two rotor turns (12 aliquots). The absolute value of the point to point difference in force was calculated and then averaged ((|F_{L-11}−F_{L-10}|+|F_{L-10}−F_{L-9}|+|F_{L-9}−F_{L-8}|+|F_{L-8}−F_{L-7}|+|F_{L-7}−F_{L-6}|+|F_{L-6}−F_{L-5}|+|F_{L-5}−F_{L-4}|+|F_{L-4}−F_{L-3}|+|F_{L-3}−F_{L-2}|+|F_{L-2}−F_{L-1}+F_{L-1}−F_{L-}|)/11). Although the standard deviation to assess the noise in the pressure response may be unnecessary to characterize noise relative to the mean, it may be implemented into the system/method. For example, the standard deviation may be implemented to further quantify trends and/or behavior in any of the pressure signal readings.

Referring to FIG. 8I, illustrates an average change in a magnitude of the pressure from aliquot to aliquot for the last two rotor turns between occluded and unoccluded. Specially, an occluded feeding set (the non-normal condition, as described and illustrated in relation to FIG. 8G and labeled in this graph as high pressure to high pressure or H2H), was compared to an unoccluded feeding set (labeled in this case as low pressure to low pressure of L2L). FIG. 8I illustrated the individual value plot of this data using 6 pumps. 4 different formula types, and 2 different feed rates. FIGS. 8J-8L illustrate the same data but broken down by pump, formula type, and feed rate, respectively.

Referring to FIG. 8M, illustrates the finalized flow chart demonstrating the decision tree for the thick formula occlusion detection algorithm after taking FIGS. 8A-8L into consideration as described above. For reference, and as described above, the C Force Value is acquired at the end of a 1-minute cycle, 5 seconds after the last aliquot is delivered. The L Force Value acquired after the last aliquot is delivered. The A Force Value is acquired immediately before the first aliquot is delivered at the start of a feed. The Avg(ΔL₁₂) is the average change in magnitude from aliquot to aliquot for the last two rotor turns.

Referring to FIG. 8N, illustrates the results of tests of the flow chart from FIG. 8M when using a variety of pumps under test, feed rates, formula types, and use conditions. 99.4% of occlusions were detected after 3 1-minute feed cycles with only 0.05% resulting in a false alarm. These results are a significant improvement over systems without using the algorithm.

While the aspects described herein have been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects, as set forth above, are intended to be illustrative, not limiting. Various changes may be made without departing from the spirit and scope of the disclosure. Therefore, the disclosure is intended to embrace all known or later-developed alternatives, modifications, variations, improvements, and/or substantial equivalents.

Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

It is understood that the specific order or hierarchy of the processes/flowcharts disclosed is an illustration of example approaches. Based upon design preferences, it is understood that the specific order or hierarchy in the processes/flowcharts may be rearranged. Further, some features/steps may be combined or omitted. The accompanying method claims present elements of the various features/steps in a sample order, and are not meant to be limited to the specific order or hierarchy presented.

Further, the word “example” is used herein to mean “serving as an example, instance, or illustration.” Any aspect described herein as “example” is not necessarily to be construed as preferred or advantageous over other aspects. Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. Nothing disclosed herein is intended to be dedicated to the public regardless of whether such disclosure is explicitly recited in the claims.

What is claimed is:

1. A method of occlusion detection of a feeding pump, the method comprising:
determining when a feeding set including a liquid is engaged in the feeding pump, wherein the liquid is a feeding formula;
advancing a first amount of the feeding formula through the feeding pump via a feeding formula conduit, wherein the first amount is a first volume of the liquid;
acquiring a first pressure sensor reading of the feeding formula conduit;
advancing a second amount of the feeding formula through the feeding pump via the feeding formula conduit, wherein the second amount is second volume of the liquid;
acquiring a second pressure sensor reading of the feeding formula conduit;
acquiring a third pressure sensor reading of the feeding formula conduit; and
detecting when an occlusion is present in the feeding formula conduit based on at least one of the first pressure sensor reading, the second pressure sensor reading and the third pressure sensor reading.

2. The method of claim 1, wherein the feeding formula is rated as level 0-4 on an International Dysphagia Diet Standardization Initiative (IDDSI) framework.

3. The method of claim 2, wherein the feeding formula is further rated as level 2-4 on the IDDSI framework.

4. The method of claim 1, further comprising pausing the feeding pump for a predetermined period of time after advancing the first amount of the feeding formula, and before performing the acquiring of the first pressure sensor reading.

5. The method of claim 4, wherein the predetermined period of time being 1-3 seconds.

6. The method of claim 4, further comprising pausing the feeding pump for a second predetermined period of time after advancing the second amount of the feeding formula and before performing the acquiring of the third pressure sensor reading.

7. The method of claim 6, wherein the pausing of the feeding pump for the second predetermined period of time is after performing the acquiring of the second pressure sensor reading.

8. The method of claim 6, wherein the second predetermined period of time being 2-7 seconds.

9. The method of claim 1, further comprising pausing the feeding pump for a predetermined period of time after advancing the second amount of the feeding formula and before performing the acquiring of the third pressure sensor reading.

10. The method of claim 9, wherein the pausing of the feeding pump for the predetermined period of time is after performing the acquiring of the second pressure sensor reading.

11. The method of claim 10, wherein the predetermined period of time being 2-7 seconds.

12. The method of claim 1, wherein the feeding formula conduit is a tube that is compressably engaged with a rotor of a feeding set.

13. The method of claim 1, wherein the first volume of the liquid and the second volume of the liquid is one aliquot.

14. The method of claim 1, wherein advancing the first amount is priming the feeding set.

15. The method of claim 1, further comprising when a difference between the third pressure sensor reading and the second pressure sensor reading is less than or equal to X pounds per square inch (PSI) the feeding formula conduit is not occluded.

16. The method of claim 1, further comprising when a difference between the third pressure sensor reading and the second pressure sensor reading is greater than X pounds per square inch, and when a difference between the third pressure sensor reading and the first pressure sensor reading is less than or equal to X₂ pounds per square inch, the feeding formula conduit is occluded.

17. The method of claim 1, further comprising when a difference between the third pressure sensor reading and the second pressure sensor reading is greater than X PSI, and when a difference between the third pressure sensor reading and the first pressure sensor reading is greater than X₂ PSI, and when an average of a plurality of previous second pressure sensor readings is greater than or equal to X₃, the feeding formula conduit is occluded.

18. The method of claim 17, wherein X is between −10 and 2 PSI, X₂ is between 0 and 10 PSI, and X₃ is between −6 and 8 PSI.

19. The method of claim 18, wherein X is between −6 and −2 PSI, X₂ is between 4 and 8 PSI, and X₃ is between −2 and 5 PSI.

20. The method of claim 19, wherein X is −4 PSI, X₂ is 5 PSI, and X₃ is 3 PSI.

21. The method of claim 17, wherein the plurality of previous second pressure sensor reading is less than or equal to 12 second pressure readings.

22. A feeding pump configured for occlusion detection comprising:
one or more memories; and
one or more processors coupled with the one or more memories and configured, individually or in combination, to:
determine when a feeding set including a liquid is engaged in the feeding pump, wherein the liquid is a feeding formula;
advance a first amount of the feeding formula through the feeding pump via a feeding formula conduit, wherein the first amount is a first volume of the liquid;
acquire a first pressure sensor reading of the feeding formula conduit;
advance a second amount of the feeding formula through the feeding pump via the feeding formula conduit, wherein the second amount is second volume of the liquid;
acquire a second pressure sensor reading of the feeding formula conduit;
acquire a third pressure sensor reading of the feeding formula conduit; and
detect when an occlusion is present in the feeding formula conduit based on at least one of the first pressure sensor reading, the second pressure sensor reading and the third pressure sensor reading.

23. A computer-readable medium comprising stored instructions for occlusion detection, wherein the instructions are executable by one or more processors, individually or in combination, to:
determine when a feeding set including a liquid is engaged in a feeding pump, wherein the liquid is a feeding formula;
advance a first amount of the feeding formula through the feeding pump via a feeding formula conduit, wherein the first amount is a first volume of the liquid;
acquire a first pressure sensor reading of the feeding formula conduit;
advance a second amount of the feeding formula through the feeding pump via the feeding formula conduit, wherein the second amount is second volume of the liquid;
acquire a second pressure sensor reading of the feeding formula conduit;
acquire a third pressure sensor reading of the feeding formula conduit; and
detect when an occlusion is present in the feeding formula conduit based on at least one of the first pressure sensor reading, the second pressure sensor reading and the third pressure sensor reading.

24. A feeding set configured for occlusion detection of a feeding pump comprising:
a liquid, wherein the liquid is a feeding formula; and
a feeding formula conduit, wherein the feeding pump is configured to advance a first amount of the feeding formula though the feeding formula conduit, and the first amount is a first volume of the liquid;
wherein the feeding pump is further configured to:
acquire a first pressure sensor reading of the feeding formula conduit;
advance a second amount of the feeding formula through the feeding pump via the feeding formula conduit, wherein the second amount is second volume of the liquid;
acquire a second pressure sensor reading of the feeding formula conduit;
acquire a third pressure sensor reading of the feeding formula conduit; and
detect when an occlusion is present in the feeding formula conduit based on at least one of the first pressure sensor reading, the second pressure sensor reading and the third pressure sensor reading.
